(19) United States
(12) Patent Application Publication
(10) Pub. No.: US 2020/0306146 A1
(43) Pub. Date: Oct. 1, 2020

(54) WET WIPE LOTION

(71) Applicant: The Procter & Gamble Company, Cincinnati, OH (US)

(72) Inventors: Randall Glenn Marsh, Hamilton, OH (US); Julia Kuhlmann, Cincinnati, OH (US)

(21) Appl. No.: 16/832,393

(22) Filed: Mar. 27, 2020

Related U.S. Application Data
(60) Provisional application No. 62/827,266, filed on Apr. 1, 2019.

(51) Int. Cl. A61K 8/02; A61K 8/9789; A61K 8/85; A61K 8/34

(52) U.S. Cl. CPC A61K 8/0208; A61K 8/9789; A61K 2800/87; A61K 8/345; A61K 2800/30; A61K 8/85

(57) ABSTRACT

Described herein is a wet wipe including a substrate and a lotion. The lotion includes from about 95% to about 99.9% water by weight of the lotion and an odor reducing composition comprising a natural extract, wherein the natural extract is selected from one of: magnolia extract, marigold extract, white tea extract, green tea powder, olive oil, green tea extract, white tea powder, and combinations thereof. The lotion has a pH from about 3.6 to about 4.8.

### CROSS REFERENCE TO RELATED APPLICATION

This application claims the benefit, under 35 U. S.C. § 119(e), of U.S. Provisional Application Ser. No. 62/827,266, filed on Apr. 1, 2019, the entire disclosure of which is incorporated herein by reference.

### FIELD

Described herein is a wet wipe comprising a substrate and a lotion, wherein the lotion comprises a natural extract, wherein the natural extract is magnolia extract and/or marigold extract.

### BACKGROUND

Wet wipes are constructed from porous or absorbent sheets impregnated with a lotion, and they are sold and stored in an air-tight container or wrapper to prevent the sheets drying out. Wet wipes are made for a variety of uses. The two main categories of use are firstly, those for general household cleaning tasks, such as the cleaning of hard surfaces like floors or kitchen surfaces and secondly, those made for personal cleansing, such as the removal of make-up, or the cleaning of infants prior to the fitting of a new diaper or the simple refreshment of the skin after meals or while traveling. Wipes have also found use with feminine health and adult incontinence products.

A major proportion of the wipes intended for the cleansing of human skin are wet wipes which are designed for use with infants and young children. Wet wipes are required to be effective at cleaning while at the same time being very gentle and mild on the skin of the baby.

Consumers are seeking more natural products, complete with fragrances that are mostly natural. Unscented or low scented products are also desired by consumers as they may be considered more natural than scented products. Thus, there is a continuing need for both natural ingredients and low odor products in the area of wet wipes.

### SUMMARY OF THE INVENTION

Described herein is a wet wipe comprising a substrate and a lotion, wherein the lotion comprises (a) from about 95% to about 99.9% water by weight of the lotion; and (b) an odor reducing composition comprising a natural extract, wherein the natural extract is magnolia extract and/or marigold extract; wherein the lotion has a pH from about 3.6 to about 4.8.

### DETAILED DESCRIPTION

The following definitions may be useful in understanding the present disclosure:

“Devoid of,” “free of,” and the like, as those terms are used herein, means that the wet wipe, lotion, and/or substrate does not have more than trace amounts or background levels of a given material, ingredient, or characteristic following these qualifiers; the amount of the material or ingredient does not cause harm or irritation that consumers typically associate with the material or ingredient; or the material or ingredient was not added to the wet wipe, lotion, and/or substrate intentionally. In some instances, “devoid of” and “free of” can mean there is no measurable amount of the material or ingredient. For example, the wet wipe in some forms contains no measurable amounts of chlorine.

“Soil” refers herein to matter that is extraneous to a surface being cleaned. For example, soils include body exudates, household matter, and outdoor matter. Body exudates include feces, menses, urine, vomitus, mucus, and the like. Household matter includes food, beverages, combinations thereof, and the like. Outdoor matter includes dirt, mud, snow, paint, crayons, and the like.

“Nonwoven” refers herein to a fibrous structure made from an assembly of continuous fibers, coextruded fibers, non-continuous fibers and combinations thereof, without weaving or knitting, by processes such as spunbonding, carding, meltblowing, airlaying, wetlaying, coforming, or other such processes known in the art for such purposes.

“Substrate” refers herein to a material which is primarily two-dimensional (i.e. in an XY plane) and whose thickness (in a Z direction) is relatively small (i.e. 1/10 or less) in comparison to the substrate's length (in an X direction) and width (in a Y direction). Non-limiting examples of substrates include a web, layer or layers or fibrous materials, nonwovens, films and foils such as polymeric films or metallic foils. These materials may be used alone or may comprise two or more layers joined together. As such, a web is a substrate.

“Wipe” refers herein to a piece of material, generally non-woven material, used in cleansing hard surfaces, food, inanimate objects, toys and body parts. In particular, many currently available wipes may be intended for the cleansing of the perianal area after defecation.

As used herein, percentages are given as the weight of the component to the total weight of the lotion, unless otherwise indicated. Percentages reflect 100% active component material. For example, if a component is available in a dispersion at a concentration of 50% component to dispersion, by weight, twice as much of the dispersion, by weight, would be added to the lotion to provide the equivalent of 100% active component.

Values disclosed herein as ends of ranges are not to be understood as being strictly limited to the exact numerical values recited. Instead, unless otherwise specified, each numerical range is intended to mean both the recited values and any integers within the range. For example, a range disclosed as “1 to 10” is intended to mean “1, 2, 3, 4, 5, 6, 7, 8, 9, 10.”

As used herein, the articles “a” and “an” when used herein, for example, “an anionic emulsifier” or “a fiber” is understood to mean one or more of the material that is claimed or described.

Wet Wipe Odor Reducing Composition

The wet wipe described herein may comprise an odor reducing composition. The odor reducing composition may be a naturally derived odor reducing composition. The naturally derived odor reducing composition may manage odors inherent to the raw materials used in the manufacture of the wet wipe and/or manage odors associated with wet wipe (e.g., urine, feces, etc.). The naturally derived odor reducing composition may include plant extracts. Exemplary plant extracts may include magnolia extract and/or marigold extract, such as Extrapone Marigold GW and Extrapone Magnolia GW from Symrise. The naturally derived odor reducing composition may be substantially scent-free.

The aqueous portion of a wet wipe, herein referred to as a lotion, is comprised of various chemical constituents that carry out functions such as cleaning, emulsification, buffering, and preservation. Each of these chemical constituents can have an inherent odor based upon its chemistry and how it was manufactured, for example what feedstocks were used and to what level the material was purified. Raw materials that are produced from plant-derived sugars, such as citric acid and sodium citrate, may have a brown-sugar smell associated with them. Raw materials derived from plant-based oils, such as sorbitan caprylate and PEG-40 hydrogenated castor oil, not surprisingly may contribute a fatty odor to the lotion. This fatty odor may be exacerbated if the lotion has a low pH, for example below 4.0, due to acid-catalyzed hydrolysis of fatty esters such as sorbitan caprylate, resulting in generation of caprylic acid over time. Caprylic acid may have an odor that is detectable at low concentrations to the human nose. When assembled together into a lotion, even a fairly small number of cosmetic raw materials can result in a final formulation that has a characteristic odor that may be noticeable and undesirable to consumers.

The nonwoven substrate component of a wipe, composed of one or more fiber types, may also contribute to the overall odor of the finished product. In the case of carded spunlaced wipes, which are typically blends of synthetic fibers such as polypropylene or polyester with cellulosic fibers such as viscose, Lyocell, or cotton, odor can arise from both the synthetic and cellulosic fibers. Synthetic fibers frequently employ spin finishes to improve their handling. Some types of spin finish chemistries are similar to surfactants with fatty tails. If the spin finishes are not washed off well during hydroentanglement, they can accumulate on the fibers and undergo degradation in the heating ovens that dry out the wet substrate. If the fatty components break down into smaller, volatile components, significant odor issues can occur. In the case of cellulosic fibers, viscose is the primary culprit. The carbon disulfide used to solubilize the cellulose to make viscose rayon usually persists at low levels in the finished fibers.

When the nonwoven is combined with the lotion into the finished wet wipe, the result is a combined odor coming from both sources and from interactions between the two. A pH below 4.0 will favor the liberation of carbon disulfide and other sulfur species from viscose rayon fibers and can generate unpleasant odor. Aging of the wipe over time, which leads to progressive lotion-substrate interactions and selective loss of some components through packaging due to their volatility, ultimately generates a particular aromatic experience for the consumer. If the wipe is unscented, this experience is typically desired to be neutral and fresh. For this reason, there is frequently the need to incorporate an odor reducing composition into the lotion to improve the neutrality of the finished product odor and boost consumer acceptance.

The present inventors have found that the addition of certain natural materials can counteract and subsequently reduce the odor of other ingredients in the wet wipe lotion. The natural materials may be added at levels so low that they themselves are not detectable, but their presence reduces the detectable odor of other materials in the lotion. Thus, only small amounts of the natural materials, such as the odor reducing composition, may be used, in some cases, from about 0.005% to about 0.05%, by weight of the lotion. In other cases, the lotion may comprise from about 0.01% to about 0.025%, by weight of the lotion, of the natural odor reducing composition. In still other cases, the lotion may comprise from about 0.015% to about 0.022%, by weight of the lotion, of the natural odor reducing composition. Natural odor reducing compositions may include, but are not limited to, magnolia extract, marigold extract, white tea extract, green tea powder, pure olive oil, green tea extract, white tea powder, and combinations thereof. Natural odor reducing compositions that perform particularly well include magnolia extract, marigold extract, white tea extract, and green tea powder.

Lotion

The wet wipe described herein comprises a lotion. The lotion described herein comprises from about 95% water to about 99.9% water, alternatively from about 98.5% water to about 99.7% water, alternatively from about 98.5% water to about 99.5% water, alternatively from about 99% water to about 99.3% water, by weight of the lotion. At such a high water content, the lotion described herein must comprise a strong preservative system for preventing microbial growth. The percent water in the lotion can be calculated using the Water Content Method provided herein.

The lotion described herein may be incorporated onto a substrate at a load of about 200% to about 600%, alternatively from about 300% to about 500%, and alternatively from about 325% to about 460%, by weight of the substrate.

The lotions described herein, and wipes comprising the lotions, may be made by the conventional processes described in the art. Alternatively, the lotions and wipes are made according to U.S. Pat. No. 7,365,030 entitled “Process for making a wet wipe using a concentrated emulsion” by Sylvie Chamba et al., which is incorporated herein by reference.

Lotion Preservative System

Controlling microbiological growth is a necessary function of wet wipes, especially wet wipes comprising a high water content lotion. To control microbiological growth, the lotion described herein comprises a preservative system. The preservative system may include a preservative enhancing agent and one or more preservatives. A preservative may be understood to be a chemical or natural compound or a combination of compounds reducing the growth of microorganisms, thus enabling a longer shelf life for a package of wet wipes (opened or not opened) as well as creating an environment with reduced growth of microorganisms when transferred to the skin during the wiping process.

The spectrum of activity of the preservative may include bacteria, molds, and/or yeast. Each of such microorganisms may be killed by the preservative. Another mode of action to be contemplated may be the reduction of the growth rate of the microorganisms without active killing. Both actions however result in a drastic reduction of the population of microorganisms.

Suitable lotion preservative systems include those described in U.S. application Ser. No. 15/892515 (Attorney Docket No. 15119), WO201905064A1, US20170367331, US20120141571, US20090155325, US20130156708, U.S. Pat. Nos. 8,969,390, 9,149,045.

An exemplary wet wipe may include a lotion emulsion composition comprising a preservative enhancing agent and a preservative. In an exemplary configuration, the lotion emulsion composition may comprise sorbitan caprylate and/or glyceryl caprylate/caprate and sodium benzoate. In a further exemplary configuration, the lotion emulsion composition may comprise sorbitan caprylate and/or glyceryl caprylate/caprate, sodium benzoate, EDTA, and a citrate-citric acid buffering system at a pH of less than about 4. Suitable preservatives and preservative system materials may be such as disclosed in U.S. Ser. Nos. 14/867,059 and 14/330,171 (Attorney Docket Nos. 13556 and 12995M). The lotion emulsion composition may include one or more preservatives. The preservative may include an organic acid or the salt thereof. Exemplary organic acids include benzoic acid or sorbic acid. Exemplary salts of organic acids include sodium benzoate and potassium sorbate, for example.

The preservative system of the lotion described herein may include additional compounds, for example chelating agents, such as ethylenediamine tetraacetic acid (EDTA) and its salts, or diethylene triamine pentaacetic acid (DTPA) and its salts, or gluconic acid and its salts. The chelating agent may be disodium EDTA or sodium gluconate.

The lotion described herein may be incorporated into a substrate at a load of about 200% to about 600%, alternatively from about 325% to about 460%, by weight of the substrate. The substrate may be a nonwoven material.

The preservative system in the lotion described herein may include one or more preservative enhancing agents. Exemplary preservative enhancing agents include sorbitan caprylate, glyceryl caprylate/caprate, or combinations thereof. An exemplary sorbitan caprylate is manufactured by Clariant under the designation VELSAN® SC. An exemplary glyceryl caprylate/caprate may be CremerCOOR® GC810, CremerCOOR® GC8, or IMWITOR® 742, all available from Peter Cremer, or CAPMUL® 708G, available from Abitec.

The lotion described herein may comprise from about 0.05% to about 0.25%, alternatively from about 0.07% to about 0.22%, alternatively from about 0.09% to about 0.2%, alternatively from about 0.11% to about 0.18%, and alternatively from about 0.14% to about 0.18% of a preservative enhancing agent by weight of the lotion.

Lotion Buffering System

The lotion described herein comprises from about 0.3% to about 1%, alternatively from about 0.4% to about 1%, alternatively from about 0.5% to about 0.85%, and alternatively from about 0.6% to about 0.75% of a pH buffering system, by weight of the lotion, which also may help prevent and/or reduce the growth of bacteria, mold, and/or yeast. The pH buffering system may be a citrate-citric acid buffering system at a pH of less than 5. The pH buffering system may be a citrate-citric acid buffering system at a pH of less than 4. The pH buffering system may be a sodium citrate-citric acid buffering system at a pH of less than 5. The pH buffering system may be a sodium citrate-citric acid buffering system at a pH of less than 4. The pH buffering system may comprise sodium citrate dihydrate and citric acid anhydrous. The lotion described herein may have a pH from about 3.5 to about 7, alternatively from about 3.5 to about 6, alternatively from about 3.6 to about 4.8, alternatively from about 3.6 to about 4.4, alternatively from about 3.6 to about 4.2, and alternatively from about 3.8 to about 4.2.

The lotion described herein may have an Equivalence Value from about 25 to about 100, alternatively from about 30 to about 95, alternatively from about 55 to about 90, alternatively from about 65 to about 90, alternatively from about 60 to about 80, alternatively from about 62 to about 75, and alternatively from about 65 to about 70, according to the Buffering Capacity Test Method described herein.

A lotion having a higher Equivalence Value, such as from about 65 to about 90, may be desired because it can help to protect a baby's skin against irritation caused by feces and urine residue that can remain after cleaning. The proteases in feces that damage the skin can start to become active around pH 5 and can reach peak activity between pH 7 to 8. Urea in urine may be converted to ammonia by bacteria on the skin, elevating the pH and making the fecal proteases more active. By depositing a lotion with a higher Equivalence Value on the skin, an acidic buffer may be created that resists the climb in pH that would normally occur over time when trace amounts of feces and urine are present. By resisting the climb in pH, a lotion with a higher Equivalence Value can protect the skin from damage caused by fecal proteases and reduce the likelihood that rash will occur between diaper changes.

The lotion described herein may be substantially free of or free of phenoxyethanol. The lotion may be substantially free of or free of isothiozolinones, formaldehydes, formaldehyde donors, and/or biguanides.

Lotion Optional Ingredients

The lotion described herein may include various optional ingredients, such as surfactants, emollients, opacifying agents, film-formers, soothing agents, skin protectants, medically active ingredients, healing actives, and the like, such as described in U.S. Pat. Nos. 7,666,827; 7,005,557; 8,221,774.

The lotion described herein may comprise an emollient. Emollients may (1) hydrate soil residues (for example, fecal residues or dried urine residues or menses residues), thus enhancing their removal from the skin, (2) hydrate the skin, thus reducing its dryness and irritation, (3) protect the skin from later irritation (for example, caused by the friction of an absorbent article) as the emollient is deposited onto the skin and remains at its surface as a thin protective layer, and (4) provide a desired sensory feel to the lotion and/or the skin.

The emollient may include plant-derived oils, silicone oils, functionalized silicone oils, hydrocarbon oils, fatty alcohols, fatty alcohol ethers, fatty acids, esters of monobasic and/or dibasic and/or tribasic and/or polybasic carboxylic acids with mono and polyhydric alcohols, polyoxyethylenes, polyoxypropylenes, mixtures of polyoxyethylene and polyoxypropylene ethers of fatty alcohols, and mixtures thereof. The emollients may be either saturated or unsaturated, have an aliphatic character and be straight or branched chained or contain alicyclic or aromatic rings.

The lotion described herein may comprise a mixture of caprylic/capric triglycerides in combination with Bis-PEG/PPG-16/16 PEG/PPG-16/16 dimethicone known as ABIL® CARE 85, available from Evonik Industries.

The lotion described herein may include one or more surfactants. The surfactant may be an individual surfactant or a mixture of surfactants. The surfactant may be a polymeric surfactant or a non-polymeric one. The surfactant or combinations of surfactants may be mild, which means that the surfactants provide sufficient cleaning or detersive benefits but do not overly dry or otherwise harm or damage the skin. The surfactant, when present in the lotion, may be present in an amount ranging from about 0.05% to about 1%, alternatively from about 0.075% to about 0.5%, alternatively from about 0.1% to about 0.2%, and alternatively from about 0.15% to about 0.2% by weight of the lotion. The surfactant may comprise PEG-40 Hydrogenated Castor Oil (PEG-40 HCO), such as EMULSOGEN® HCW049 manufactured by Clariant. The surfactant may comprise a polyglyceryl ester or a mixture of polyglyceryl esters.

The lotion described herein may comprise one or more rheology modifiers. A rheology modifier may help to stabilize the lotion by reducing or preventing coalescence of droplets of the hydrophobic materials in the composition.

Non-limiting examples of rheology modifiers include, but are not limited to, hydrocolloids, including natural gums, such as xanthan gum. The lotion described herein may comprise from about 0.01% to about 0.1%, alternatively from about 0.03% to about 0.08%, alternatively from about 0.05% to about 0.07%, and alternatively about 0.06% of a rheology modifier by weight of the lotion.

The wet wipe and/or lotion described herein may be devoid of fragrance and/or perfume.

Wet Wipe Substrate

The lotion described herein may be loaded onto a substrate to form a wet wipe. The substrate may be a nonwoven material. The nonwoven material may comprise one or more layers of fibrous structures, wherein each layer may include continuous fibers, coextruded fibers, non-continuous fibers, and combinations thereof.

The substrate described herein comprises from about 5% to about 100%, alternatively from about 15% to about 95%, alternatively from about 25% to about 90%, alternatively from about 35% to about 90%, alternatively from about 45% to about 85%, alternatively from about 50% to about 80%, alternatively from about 55% to about 75%, alternatively from about 65% to about 75% polyester, by weight of the substrate. The polyester may comprise less than 100 ppm antimony or the polyester may be devoid of antimony.

The substrate described herein comprises from about 14.5% to about 100%, alternatively from about 14.5% to about 95%, alternatively from about 14.5% to about 75%, alternatively from about 14.5% to about 45%, alternatively from about 15% to about 40%, alternatively from about 15% to about 25%, alternatively from about 0.1% to about 10%, and alternatively from about 15% to about 20% cotton, by weight of the substrate. The cotton may be devoid of chlorine. The cotton may be organic cotton. The cotton may not be bleached. The cotton may be free or substantially free of visual foreign matter, including cotton leaves, stalk, and/or other plant matter that has not been fully removed from the cotton fiber. The average visual foreign matter (VFM) <9 mm² per linear meter of the substrate may be from about 0.001 VFM to about 0.05 VFM, alternatively from about 0.005 VFM to about 0.03 VFM, alternatively from about 0.007 VFM to about 0.02 VFM, and alternatively from about 0.009 VFM to about 0.015 VFM, when the width of the substrate is 3 meters, using a web inspection vision system which may be available from Cognex.

The substrate described herein may comprise from about 5% to about 50%, alternatively from about 10% to about 30%, alternatively from about 5% to about 25%, alternatively from about 10% to about 20%, and alternatively about 15% viscose, by weight of the substrate.

The substrate described herein may comprise from about 14.5% to about 20% cotton by weight of the substrate, from about 10% to about 20% viscose by weight of the substrate, and from about 60% to about 80% polyester by weight of the substrate.

Any additional fibers of the substrate may be comprised of any natural, cellulosic, and/or wholly synthetic material. The substrate may comprise from about 5% to about 85%, alternatively from about 10% to about 65%, alternatively from about 15% to about 45%, alternatively from about 20% to about 35%, alternatively from about 0.1% to about 30%, alternatively from about 0.5% to about 5%, and alternatively from about 0.01% to about 1% of the additional fibers, by weight of the substrate. Examples of natural fibers may include cellulosic natural fibers, such as fibers from hardwood sources, softwood sources, or other non-wood plants. The natural fibers may comprise cellulose, starch and combinations thereof. Non-limiting examples of suitable cellulosic natural fibers include wood pulp, typical northern softwood Kraft, typical southern softwood Kraft, typical CTMP, typical deinked, corn pulp, acacia, eucalyptus, aspen, reed pulp, birch, maple, radiata pine and combinations thereof. Other sources of natural fibers from plants include albardine, esparto, wheat, rice, corn, sugar cane, papyrus, jute, reed, sabia, raphia, bamboo, sidal, kenaf, abaca, sunn, rayon (also known as viscose), lyocell, cotton, hemp, flax, ramie and combinations thereof. Yet other natural fibers may include fibers from other natural non-plant sources, such as, down, feathers, silk, cotton and combinations thereof. The natural fibers may be treated or otherwise modified mechanically or chemically to provide desired characteristics or may be in a form that is generally similar to the form in which they may be found in nature. Mechanical and/or chemical manipulation of natural fibers does not exclude them from what are considered natural fibers with respect to the development described herein.

The synthetic fibers may be any material, such as those selected from the group consisting of polyesters (e.g., polyethylene terephthalate), polyolefins, polypropylenes, polyethylenes, polyethers, polyamides, polyesteramides, polyvinylalcohols, polyhydroxyalkanoates, polysaccharides, and combinations thereof. Further, the synthetic fibers may be a single component (i.e., single synthetic material or mixture makes up entire fiber), bi-component (i.e., the fiber is divided into regions, the regions including two or more different synthetic materials or mixtures thereof and may include co-extruded fibers and core and sheath fibers) and combinations thereof. Bicomponent fibers may be used as a component fiber of the structure, and/or they may be present to act as a binder for the other fibers present in the fibrous structure. Any or all of the synthetic fibers may be treated before, during, or after manufacture to change any desired properties of the fibers. The substrate may comprise hydrophilic fibers, hydrophobic fibers, or a combination thereof.

The substrate may comprise various percentages of natural and/or synthetic fibers. For example, the substrate may comprise 100% synthetic fibers. The substrate may comprise natural and synthetic fibers. For example, the substrate may comprise from about 0% to about 95% natural fibers, with the balance comprising synthetic fibers.

It may be desirable to have particular combinations of fibers to provide desired characteristics. For example, it may be desirable to have fibers of certain lengths, widths, coarseness or other characteristics combined in certain layers, or separate from each other. The fibers may be of virtually any size and may have an average length from about 1 mm to about 60 mm. Average fiber length refers to the length of the individual fibers if straightened out. The fibers may have an average fiber width of greater than about 5 micrometers. The fibers may have an average fiber width of from about 5 micrometers to about 50 micrometers. The fibers may have a coarseness of greater than about 5 mg/100 m. The fibers may have a coarseness of from about 5 mg/100 m to about 75 mg/100 m.

The fibers may be circular in cross-section, dog-bone shape, delta (i.e., triangular cross section), trilobal, ribbon, or other shapes typically produced as staple fibers. Likewise, the fibers may be conjugate fibers such as bicomponent fibers. The fibers may be crimped and may have a finish, such as a lubricant, applied.

The substrate materials may also be treated to improve the softness and texture thereof. The substrate may be subjected to various treatments, such as physical treatment, hydro-molding, hydro-embossing, and ring rolling, as described in U.S. Pat. No. 5,143,679; structural elongation, as described in U.S. Pat. No. 5,518,801; consolidation, as described in U.S. Pat. Nos. 5,914,084; 6,114,263; 6,129,801 and 6,383,431; stretch aperturing, as described in U.S. Pat. Nos. 5,628,097; 5,658,639; and 5,916,661; differential elongation, as described in U.S. Pat. No. 7,037,569, and other solid state formation technologies as described in U.S. Pat. Nos. 7,553,532 and 7,410,683; zone activation, and the like; chemical treatment, such as rendering part or all of the substrate hydrophobic, and/or hydrophilic, and the like; thermal treatment, such as thermal-embossing, softening of fibers by heating, thermal bonding and the like; and combinations thereof.

Without wishing to be bound by theory, it is believed that a textured substrate may further enable the ease of removal of soils by improving the ability to grip or otherwise lift the soils from the surface during cleansing. Any one of a number of texture elements may be useful in improving the ability to grip or otherwise lift the soil from the surface during cleansing such as continuous hydro-molded elements, hollow molded element, solid molded elements, circles, squares, rectangles, ovals, ellipses, irregular circles, swirls, curly cues, cross hatches, pebbles, lined circles, linked irregular circles, half circles, wavy lines, bubble lines, puzzles, leaves, outlined leaves, plates, connected circles, changing curves, dots, honeycombs, and the like, and combinations thereof. The texture elements may be hollow elements. The texture elements may be connected to each other. The texture elements may overlap each other.

The substrate may have a basis weight between about 15, 30, 40, 45 or 52 g/m² and about 65, 75, 85, 95, or 100 g/m². Exemplary nonwoven substrates are described in U.S. Patent Publication 2012/066852 and U.S. Patent Publication U.S. 2011/244199.

The surface of the substrate may be essentially flat. The surface of the substrate may optionally contain raised and/or lowered portions. The raised and/or lowered portions may be in the form of logos, indicia, trademarks, geometric patterns, and/or images of the surfaces that the substrate is intended to clean (i.e., infant's body, face, etc.). The raised and/or lowered portions may be randomly arranged on the surface of the substrate or be in a repetitive pattern of some form.

The substrate may be biodegradable. For example, the substrate could be made from a biodegradable material such as a polyesteramide, or a high wet strength cellulose. The substrate may be dispersible.

The wet wipes described herein may have different properties on different sides of the wet wipe. For example, one side of the wipe may have good cleaning performance and the other side of the wet wipe may have good tactile sensation to the user. In another example, one side of the wet wipe may have an increased cleaning performance as compared to the other side of the wet wipe.

Non-limiting examples of processes for making fibrous structure of the substrate described herein include known wet-laid papermaking processes, air-laid papermaking processes including carded and/or spunlaced processes. Such processes typically include steps of preparing a fiber composition in the form of a suspension in a medium, either wet, more specifically aqueous medium, or dry, more specifically gaseous, i.e. with air as medium. The aqueous medium used for wet-laid processes is oftentimes referred to as a fiber slurry. The fibrous slurry is then used to deposit a plurality of fibers onto a forming wire or belt such that an embryonic fibrous structure is formed, after which drying and/or bonding the fibers together results in a fibrous structure. Further processing the fibrous structure may be carried out such that a finished fibrous structure is formed. For example, in typical papermaking processes, the finished fibrous structure is the fibrous structure that is wound on the reel at the end of papermaking, and may subsequently be converted into a finished product, e.g. a sanitary tissue product.

The fibrous structures of the substrate described herein may be homogeneous or may be layered. If layered, the substrate may comprise at least two and/or at least three and/or at least four and/or at least five layers.

The fibrous structure of the substrate described herein may be a co-formed fibrous structure.

“Co-formed fibrous structure” as used herein means that the fibrous structure comprises a mixture of at least two different materials wherein at least one of the materials comprises a filament, such as a polypropylene filament, and at least one other material, different from the first material, comprises a solid additive, such as a fiber and/or a particulate. In one example, a co-formed fibrous structure comprises solid additives, such as fibers, such as wood pulp fibers and/or absorbent gel materials and/or filler particles and/or particulate spot bonding powders and/or clays, and filaments, such as polypropylene filaments.

“Solid additive” as used herein means a fiber and/or a particulate.

“Particulate” as used herein means a granular substance or powder.

“Fiber” and/or “Filament” as used herein means an elongate particulate having an apparent length greatly exceeding its apparent width, i.e. a length to diameter ratio of at least about 10. For purposes of the present disclosure, a “fiber” is an elongate particulate as described above that exhibits a length of less than 5.08 cm (2 in.) and a “filament” is an elongate particulate as described above that exhibits a length of greater than or equal to 5.08 cm (2 in.).

The substrate described herein may be subjected to any post-processing operations such as embossing operations, printing operations, tuft-generating operations, thermal bonding operations, ultrasonic bonding operations, perforating operations, surface treatment operations such as application of lotions, silicones and/or other materials, folding, and combinations thereof.

Article of Commerce

An article of commerce may comprise a package and a plurality of the wet wipes described herein.

The package may comprise one or more communications that the wet wipe and/or substrate described herein comprises an odor reducing composition, comprises an odor reducing composition comprising a natural extract, and/or comprises an odor reducing composition comprising a natural extract of magnolia extract and/or marigold extract. The package may comprise one or more communications that the wet wipe and/or the substrate described herein comprises cotton and/or is devoid of chlorine. The package may comprise a seal of cotton trademarked logo. The package may comprise one or more communications that the wet wipe and/or the lotion described herein is devoid of fragrance and/or perfume. The package may comprise one or more communications that the wet wipe and/or the lotion described herein comprises 99% water. The package may comprise one or more communications that the wet wipe and/or the substrate described herein is devoid of phthalate. The package may comprise one or more communications that the wet wipe and/or the lotion described herein is devoid of alcohol and/or comprises a plant extract. The package may comprise one or more communications that the wet wipe and/or lotion described herein is hypoallergenic. The package may comprise one or more communications that the wet wipe and/or lotion described herein is devoid of phenoxyethanol. The package may comprise one or more communications that the wet wipe and/or lotion described herein is “natural” or “all natural”.

The package may be in the form of a container. Containers may include, but are not limited to, tubs, flow wrap pouches, individual sachets, chained sachets comprising a tear line between each sachet, and other forms known in the art as suitable for storing nonwoven articles. Additionally, the container may also be manufactured to facilitate removal of individual wet wipes.

The container may be made of any suitable material or materials and may be manufactured in any suitable manner. For example, the container may be made of polystyrene, polypropylene, PET, POET, polyethylene, polyester, polyvinyl alcohol, or the like. The container may also be made of a mixture of the above materials. The container may be made of a metal foil. The container may be manufactured by, for example, a vacuum molding process or an injection molding process, or any suitable process.

Additional information on containers, as well as additional optional components for containers, including, but not limited to: container bodies, lids, container features, such as, but not limited to, attachment of lids, hinges, zippers, securing aids, and the like, may be found in U.S. Pat. Nos. Des. 451,279; Des. 437,686; Des. 443,508; Des 443,451; Des 421,901; Des 421,902; Des 416,794; Des 414,637; Des 445,329; U.S. Pat. Nos. 3,982,659; 3,967,756; 3,986,479; 3,994,417; 6,269,970; 5,785,179; 5,366,104; 5,322,178; 5,050,737; 4,971,220; 6,296,144; 6,315,114; 4,840,270; 4,471,881; 5,647,506; 6,401,968; 6,269,969; 6,412,634; 5,791,465; 6,092,690; U.S. Patent Application Publication No. 2002/0064323 published on May 30, 2002, issued to Chin; and WO 00/27268 published on May 18, 2000 and assigned to The Procter & Gamble Company; WO 02/14172 published on Feb. 21, 2002 and assigned to The Procter & Gamble Company; and WO 99/55213 published on Nov. 4, 1999 and assigned to The Procter & Gamble Company.

Test Methods

All test methods are carried out in an environment 23±2° C. and 50±5% relative humidity unless otherwise specified.

### Malodor Test Method

This method is used to measure the absolute intensity of a product, air sample, or substrate (fabric, blotter, cloth, etc.) at any stage in its performance profile by qualified trained expert odor evaluators. The expert odor evaluators should be screened for olfactive acuity, trained on how to discriminate intensity and character, and should regularly calibrate vs. each other. A minimum of two expert odor evaluators should perform the evaluation. The control should be prepared in the same way that the test samples are prepared for comparison. Samples for evaluation should be coded so as not to be obvious what is being graded in order to eliminate bias. Samples should be evaluated one by one with an odor grade being assigned after each evaluation.

Inhale slowly to assess odor character and intensity. Using the scale below, record the odor intensity grade. Additionally, you may enter comments about character. The odor intensity scale is a 0-100 point scale. The smallest measurable difference on this scale is 2.5 points. Recorded odor grades are a mathematical average of all grades rounded to the nearest 2.5 point. If the two grades given in which the average is directly between two points on the scale (i.e. 27.5 and 30 with average of 28.75) the final grade will be rounded down to 27.5. If there is more than a 5 point difference between two graders, a third qualified odor grader needs to evaluate the sample and the resulting grade is an average of all three.

The 0-100 Odor Intensity Scale: 100 very strong odor; 75 strong odor; 50 moderate odor; 25 slight odor; 0 no detectable odor.

### Lotion Expression Method

Lotion is expressed from wet wipes for further analysis using the Lotion Expression Method. In this method, lotion is extracted from one or more like packages of lotioned wipes that have been wetted in a sealed package for at least 28 days at 40° C. and 75% relative humidity in order to collect a desired quantity of lotion for further analysis.

One corner is cut from one sealed wipes package to create a small (approximately 0.25-inch in diameter) hole from which liquid can escape. The package is then positioned between the platens of a uniaxial press with the corner containing the hole for liquid escape hanging outside the edge of the platens such that lotion can be collected in a suitable container as pressure is applied. Pressure is then increasingly applied to the package of wipes and held at 100±10 psi for at least a minute and until no more fluid drains freely from the compressed lotion package, at which point the container holding the collected lotion is sealed tightly until further analysis. This process is repeated until the desired quantity of lotion for further analysis has been collected. If lotion is collected from more than one package of like wipes, all lotion expressed from all like packages is mixed prior to any analysis.

If lotioned wipes are packaged directly in packaging that is not amenable to the pressure/deformation required by this method, lotioned wipes are removed from packaging and are immediately transferred to a clean rectangular plastic bag and sealed. The rectangular plastic bag is then cut at one corner and placed in the platens of a press and squeezed as specified above.

### Water Content Method

In the Water Content Method, a portion of expressed lotion is placed in an oven to facilitate evaporation, and the remaining unevaporated mass is measured. From this, the water content of the starting expressed lotion is calculated.

The Lotion Expression Method is used to express lotion, from which a 5.0±0.1 g aliquot is taken and placed in a 70-mm diameter aluminum weighing boat (such as VWR part number 25433-089, VWR International, Radmor, Pa., USA, or equivalent), and the initial mass of the lotion aliquot is determined to at least the nearest 0.001 g. Immediately following weighing, the weighing boat containing the lotion is placed in an oven held at 100° C. for 12±1 hour, at which point the boat containing the unevaporated material remaining from the aliquot is removed, and the mass of unevaporated material is determined to the nearest 0.001 g. The quotient of the unevaporated mass remaining from the lotion aliquot to the initial mass of the lotion aliquot, expressed as a percent to the nearest tenth of a percent, is defined as the Lotion Percent Solids Parameter. The Lotion Percent Solids Parameter is subtracted from 100.0%, and the resulting difference is defined as the Lotion Percent Water Parameter.

### Buffering Capacity Test Method

In the Buffering Capacity Test Method, the mass of base solution required to bring a specified mass of lotion to a characteristic equivalence point is measured and expressed as a ratio of mass of added base to initial mass of lotion, and this dimensionless ratio is defined as the Equivalence Value.

The Lotion Expression Method is used to express 40.0±0.1 g of lotion. The pH of the lotion is measured to the nearest 0.01 unit of pH with a pH meter. If the pH of the lotion is equal to or greater than 10.00, the Equivalence Value is defined as 0. Otherwise, 0.1 N sodium hydroxide is added to the initial 40.0±0.1 g of expressed lotion with stirring and pH is measured and recorded as a function of added volume of base to the nearest 0.01 until the pH reaches 10.00. The pH versus mass 0.1 N sodium hydroxide added data are then examined, and the mass of added base at which the rate of change of pH with respect to addition of base is at a maximum is found and recorded in grams to the nearest 0.01 g as the Equivalence Mass. The Equivalence Mass divided by the initial mass of expressed lotion (40.0±0.1 g), then multiplied by 100, is defined as the Equivalence Value, reported to the hundredths place in dimensionless units.

### Substrate Cellulosic Fiber Content Test Method

The content of cellulosic fibers (e.g. cotton, viscose rayon) of the wet wipe substrate is determined using common methods known to those of skill in the art to both identify the identity of fibers present and to choose an appropriate method for relative quantification. For example, ISO 1833-1977 can be used to quantify a variety of common mixtures of fibers, and in particular, section 10, Mixtures of Cellulose and Polyester Fibres, can be used to determine the content of cellulosic fibers in the presence of polyester fibers.

### EXAMPLES & DATA

The following examples are provided to help illustrate the lotions described herein. The exemplified lotions may be prepared by conventional formulation and mixing techniques. It will be appreciated that other modifications of the lotions described herein, within the skill of those in the formulation art, may be undertaken. All parts, percentages, and ratios herein are by weight unless otherwise specified. Some components may come from suppliers as dilute solutions. The amount stated reflects the weight percent of the active material, unless otherwise specified.

Exemplary lotion formulations are shown in Table 1.

TABLE 1

| Ingredient | Ex. 1 | Ex. 2 | Ex. 3 | Ex. 4 | Ex. 5 |
|---|---|---|---|---|---|
| Water | Q.S. | Q.S. | Q.S. | Q.S. | Q.S. |
| PEG-40 HCO | 0.20 | 0.22 | 0.20 | 0.44 | 0.44 |
| (10% water) | | | | | |
| Odor Reducing | 0.02 | 0.02 | 0.01 | 0.02 | 0.01 |
| Composition | | | | | |
| Sorbitan Caprylate | 0.18 | 0.20 | 0.18 | 0.20 | 0.20 |
| Trisodium Citrate | 0.26 | 0.26 | 0.26 | 0.32 | 0.32 |
| Dihydrate | | | | | |
| Sodium Benzoate | 0.20 | 0.22 | 0.20 | 0.20 | 0.20 |
| Disodium EDTA | 0.10 | 0.10 | 0.10 | 0.15 | 0.15 |
| Citric Acid | 0.54 | 0.54 | 0.54 | 0.61 | 0.61 |
| Xanthan Gum | 0 | 0 | 0 | 0.06 | 0.06 |
| Abil Care 85 | 0 | 0 | 0 | 0.1 | 0.1 |
| pH | 3.8 | 3.8 | 3.8 | 3.8 | 3.8 |

1. Create a premix of PEG-40 hydrogenated castor oil and sorbitan caprylate, mixing with an IKA ULTRA-TURRAX® (for example model T-50 with a S 50 N-W 80 SMK jet mixer head) or equivalent mixer for 10 minutes at 5000-6000 rpm. Set aside.

2. Add distilled, deionized water to the mixing vessel and start the mixing with the same mixer and speed as in step 1, maintaining this mixing throughout the entire process.

3. Add trisodium citrate dihydrate, mix 15 seconds, add sodium benzoate, mix 1 minute, add disodium EDTA, mix 1 minute.

4. Add PEG-40 hydrogenated castor oil and sorbitan caprylate premix slowly to the mixing vessel, taking 15-20 seconds to add and allow for incorporation. Mix for 2 minutes.

5. Add citric acid anhydrous.

6. Add odor reducing composition.

7. Mix for 10 minutes.

8. Stop mixing. Confirm final pH is within desired range, e.g. target pH±0.1.

Table 2 shows the odor intensity grade and comments for finished wipes for a control and Examples A-G of potential odor reducing compositions, as determined by the Malodor Test Method. The control was Example 1 minus its odor reducing composition, combined with a commercially available Aqua Pure high PET substrate. Examples A-G were wipes with lotions and substrates made from Example 1 with each respective odor reducing composition. All odor reducing compositions, other than olive oil, are available from Symrise, as Extrapone Magnolia GW (liquid extract), Extrapone Marigold GW (liquid extract), Neo Extrapone Organic White Tea (powder), Actipone Organic White Tea (liquid extract), Neo Actipone Organic Green Tea (powder), and Extrapone Green Tea (liquid extract), respectively. The odor reducing compositions may be about 90% glycerin and water, with about 10% extract. Olive oil is pure olive oil. Each liquid, powder or oil is the odor reducing composition listed in the table.

The lotions were then put onto identical substrates at a lotion load of 450% and submitted to expert odor evaluators as described in the Malodor Test Method above.

TABLE 2

| | | Odor | | |
|---|---|---|---|---|
| Product | Odor | reducing | Odor | |
| Descrip- | reducing | composition | Inten- | Com- |
| tion | composition | Level (%) | sity | ments |
| Control¹ | N/A | 0 | 15 | Slight |
| | | | | waxy; |
| | | | | Sour |
| Example | Magnolia | 0.02 | 0 | Neutral |
| A | Extract | | | |
| Example | Marigold | 0.02 | 0 | Neutral |
| B | Extract | | | |
| Example | Organic | 0.02 | 10 | Slight |
| C | White Tea | | | Waxy/ |
| | Powder | | | Fatty |
| Example | Organic White | 0.02 | 0 | Slight |
| D | Tea Extract | | | Papery |
| Example | Pure Olive | 0.02 | 5 | Slight |
| E | Oil | | | Fatty |
| Example | Organic Green | 0.02 | 0 | Slight |
| F | Tea Powder | | | Papery |
| Example | Green Tea | 0.02 | 5 | Slight |
| G | Extract | | | Waxy |

As can be seen, the samples with the odor reducing compositions had a lower odor intensity than the control. High performing odor reducing compositions include the magnolia extract and the marigold extract, for which the odor intensity was 0 and the odor was neutral.

### Additional Examples/Combinations

A. A wet wipe comprising a substrate and a lotion, wherein the lotion comprises
   a) from about 95% to about 99.9% water by weight of the lotion;
   b) an odor reducing composition comprising a natural extract, wherein the natural extract is magnolia extract and/or marigold extract; and
   c) a pH buffering system;
   wherein the lotion has a pH from about 3.6 to about 4.8.
B. The wet wipe according to paragraph A, wherein the substrate further comprises about 14.5% to about 45% cotton by weight of the substrate.
C. The wet wipe according to paragraph B, wherein the cotton is devoid of chlorine.
D. A package comprising the wet wipe according to paragraph C, wherein the package comprises one or more communications that the substrate comprises cotton and/or is devoid of chlorine.
E. The wet wipe according to any of paragraphs A-D, wherein the lotion is devoid of fragrance and/or perfume.
F. A package comprising the wet wipe according to paragraph E, wherein the package comprises one or more communications that the lotion is devoid of fragrance and/or perfume.
G. A package comprising the wet wipe according to any of paragraphs A-F, wherein the package comprises one or more communications that the lotion comprises 99% water.
H. A package comprising the wet wipe according to paragraph B, wherein the package comprises a seal of cotton trademarked logo.
I. The wet wipe according to any of paragraphs A-H, wherein the naturally derived reducing composition is substantially scent-free.
J. A wet wipe comprising a substrate and a lotion, wherein the substrate comprises from about 55% to about 85% polyester by weight of the substrate, wherein the substrate comprises from about 14.5% to about 45% cotton by weight of the substrate, wherein the lotion comprises from about 98.5% to about 99.5% water by weight of the lotion, and wherein the lotion has an Equivalence Point from about 10 to about 50.
K. The wet wipe according to paragraph L, wherein the cotton is devoid of chlorine.
L. A package comprising the wet wipe according to paragraph M, wherein the package comprises one or more communications that the substrate comprises cotton and/or is devoid of chlorine.
M. The wet wipe according to any of paragraphs L-N, wherein the lotion is devoid of fragrance and/or perfume.
N. A package comprising the wet wipe according to paragraph O, wherein the package comprises one or more communications that the lotion is devoid of fragrance and/or perfume.
O. A package comprising the wet wipe according to any of paragraphs L-P, wherein the package comprises a seal of cotton trademarked logo.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A wet wipe comprising a substrate and a lotion, wherein the lotion comprises:
a) from about 95% to about 99.9% water by weight of the lotion; and
b) an odor reducing composition comprising a natural extract, wherein the natural extract is magnolia extract and/or marigold extract;
wherein the lotion has a pH from about 3.6 to about 4.8.

2. The wet wipe of claim 1, wherein the substrate further comprises about 14.5% to about 45% cotton by weight of the substrate.

3. The wet wipe of claim 2, wherein the cotton is devoid of chlorine.

4. A package comprising the wet wipe of claim 3, wherein the package comprises one or more communications that the substrate comprises cotton and/or is devoid of chlorine.

5. The wet wipe of claim 1, wherein the substrate comprises from about 55% to about 85% polyester by weight of the substrate.

6. The wet wipe of claim 1, wherein the lotion is devoid of fragrance and/or perfume. A package comprising the wet wipe of claim 6, wherein the package comprises one or more communications that the lotion is devoid of fragrance and/or perfume.

8. A package comprising the wet wipe claim 1, wherein the package comprises one or more communications that the lotion comprises 99% water.

9. The wet wipe of claim 1, wherein the odor reducing composition comprises glycerin, water, and the natural extract.

10. The wet wipe of claim 1, wherein the wet wipe is devoid of perfume.

11. The wet wipe of claim 1, wherein the wet wipe has an Odor Intensity of between 0 and about 10, according to the Malodor Test Method.

12. The wet wipe of claim 1, wherein the lotion comprises a pH buffering system.

13. The wet wipe of claim 1, wherein the lotion further comprises sorbitan caprylate.

14. The wet wipe of claim 1, where the lotion comprises from about 0.005% to about 0.05%, by weight of the lotion, of the odor reducing composition.

15. A wet wipe comprising a substrate and a lotion, wherein the lotion comprises:
a) from about 95% to about 99.9% water by weight of the lotion; and
b) an odor reducing composition comprising a natural extract, wherein the natural extract is magnolia extract and/or marigold extract;
wherein the lotion has a pH from about 3.6 to about 4.8; and
wherein the lotion is free of preservatives.

16. The wet wipe of claim 15, wherein the lotion is devoid of fragrance and/or perfume.

17. The wet wipe of claim 15, wherein the wet wipe has an Odor Intensity of between 0 and about 10, according to the Malodor Test Method.

18. The wet wipe of claim 15, wherein the substrate further comprises about 14.5% to about 45% cotton by weight of the substrate.

19. The wet wipe of claim 18, wherein the cotton is devoid of chlorine.

20. A wet wipe comprising a substrate and a lotion, wherein the lotion comprises:
a) from about 95% to about 99.9% water by weight of the lotion; and
b) an odor reducing composition comprising a natural extract, wherein the natural extract is selected from the group consisting of magnolia extract, marigold extract, white tea extract, green tea powder, olive oil, green tea extract, white tea powder, and combinations thereof;
wherein the lotion has a pH from about 3.6 to about 4.8.
